(19) United States
(12) Patent Application Publication
(10) Pub. No.: US 2022/0347378 A1
(43) Pub. Date: Nov. 3, 2022

(54) Disposable Wearable Micro-Flow Continuous Drug Delivery System

(71) Applicant: SHANGHAI XURUN MEDICAL TECHNOLOGY CO., LTD, Shanghai (CN)

(72) Inventors: Xin XI, Shanghai (CN); Bin XI, Shanghai (CN); Mingsheng CHEN, Shanghai (CN)

(21) Appl. No.: 17/598,858

(22) PCT Filed: Nov. 20, 2020
(86) PCT No.: PCT/CN2020/130613

§ 371 Date: Sep. 27, 2021

(30) Foreign Application Priority Data
Jan. 3, 2020 (CN) 202010005155.X

(51) Int. Cl. A61M 5/148; A61M 5/168; A61M 5/142; A61J 1/14

(52) U.S. Cl. CPC A61M 5/148; A61M 5/16881; A61M 5/14244; A61J 1/1475

(57) ABSTRACT

A disposable wearable micro-flow continuous drug delivery system has an elastic drug storage cavity for extruding and storing liquid drug, wherein one end of the elastic drug storage cavity is communicated with a drug outlet pipe, wherein one end of the drug outlet pipe is communicated with a first micro-flow path and a second micro-flow path for shunting liquid drug, which is far away from the elastic drug storage cavity, wherein an adjusting mechanism is connected between the first micro-flow path and a second micro-flow path, wherein a first valve and a second valve are respectively provided at the surface of the first micro-flow path and adjacent to two different sides of the adjusting mechanism, wherein the disposable wearable micro-flow continuous drug delivery system of the present invention makes the liquid drug have a certain impulse by using the elastic and soft drug storage cavity, which is convenient to inject the liquid drug into the human body, and secondly, the adjusting mechanism is set between the two micro-flow paths, and a plurality of micro valves are set on the two micro-flow paths respectively, which enable the injection volume of the liquid drug to be controlled by controlling the switches of the micro valves located at different positions to help facilitate medical treatment and minimize the effect on the daily living activity of patients.

### BACKGROUND OF THE PRESENT INVENTION

### Field of Invention

The present invention relates to the technical field of medical devices, and more particularly to a disposable wearable micro-flow continuous drug delivery system.

### Description of Related Arts

A micro-flow continuous drug delivery system is a device for injecting a drug into the human body in a continuous micro-flow. However, with the development of science and technology, people have higher and higher requirements for micro-flow continuous drug delivery system, resulting in the traditional micro-flow continuous drug delivery system has been unable to meet the needs of people.

At present, the continuous drug delivery system with extremely low flow rate and adjustable drug delivery speed is driven by micro high-precision servo motor controlled by an electronic circuit, and has the following disadvantages:

Firstly, it has a high use cost: tens of thousands of yuan are needed to buy a portable device, and then a certain amount of money is needed to buy disposable syringe and pipeline system, so more capital investments are needed when it is used.

Secondly, it is inconvenient to use: when the existing equipment is in use, it needs a catheter through multi-layer clothes to connect the infusion equipment and the injection needle, and when the clothes are thin, there is no place to carry the infusion equipment.

Thirdly, it cannot be used when in bathing and some types of sports. Therefore, we propose a disposable wearable micro-flow continuous drug delivery system.

### SUMMARY OF THE PRESENT INVENTION

The present invention is advantageous in that it provides a disposable wearable micro-flow continuous drug delivery system, which integrates and miniaturizes the control system, the syringe, the pipeline and the injection needle thereof to enable it to be attached on the skin of the injection site, so as to avoid affecting the life and movement of a user.

In order to achieve the above purpose, the technical solution adopted by the present invention is as follows:

A disposable wearable micro-flow continuous drug delivery system having an elastic drug storage cavity for extruding and storing liquid drug, wherein one end of the elastic drug storage cavity is communicated with a drug outlet pipe, wherein one end of the drug outlet pipe is communicated with a first micro-flow path and a second micro-flow path for shunting liquid drug, which is far away from the elastic drug storage cavity, wherein an adjusting mechanism is connected between the first micro-flow path and a second micro-flow path, wherein a first valve and a second valve are respectively provided at the surface of the first micro-flow path and adjacent to two different sides of the adjusting mechanism, a third valve and a fourth valve are respectively provided at the surface of the second micro-flow path and adjacent to the two different sides of the adjusting mechanism, wherein one end of the first micro-flow path is communicated with one end of the second micro-flow path, which are far away from the elastic drug storage cavity and define an outlet, wherein the adjusting mechanism has a piston chamber provided between the first micro-flow path and the second micro-flow path and a piston movably installed in the piston chamber.

Preferably, the first valve is located on the side of the second valve, which is close to the elastic drug storage cavity, and the third valve is located on the side of fourth valve, which is close to the elastic drug storage cavity.

Preferably, the adjusting mechanism further comprises a spherical cavity provided between the first micro-flow path and the second micro-flow path and an elastic spacing layer movably installed at the center of the spherical cavity.

Preferably, two ends of the adjusting mechanism are respectively communicated with the first micro-flow path and the second micro-flow path.

Compared with the prior art, the present invention has the following beneficial effects:

By using the elastic and soft drug storage cavity to give the liquid a pressure, so as to make the liquid drug have a certain impulse force, which not only facilitates the injection of the liquid drug into the human body, but also makes it easier to carry. Secondly, the adjusting mechanism is disposed between the two micro-flow paths, and a plurality of micro-valves are disposed in the two micro-flow paths respectively so that the injection volume of the liquid drug can be controlled by controlling on-off of the micro-valves at different positions, which is convenient for patients and will minimize the impact on patients' life activities.

### BRIEF DESCRIPTION OF THE DRAWINGS

FIG. 1 is a schematic diagram of a disposable wearable micro-flow continuous drug delivery system of the present invention, wherein the adjusting mechanism of disposable wearable micro-flow continuous drug delivery system has a piston chamber and a piston.

FIG. 2 is a schematic diagram of a disposable wearable micro-flow continuous drug delivery system of the present invention, wherein the adjusting mechanism of disposable wearable micro-flow continuous drug delivery system has a spherical cavity and an elastic spacing layer.

In the Figs, the reference number 1 represents the elastic drug storage cavity, the reference number 2 represents the first valve, the reference number 3 represents the second valve, the reference number 4 represents the third valve, the reference number 5 represents the fourth valve, the reference number 6 represents the outlet, the reference number 7 represents the piston chamber, the reference number 8 represents the piston, the reference number 9 represents the spherical cavity, and the reference number 10 represents the elastic spacing layer.

### DETAILED DESCRIPTION OF THE PREFERRED EMBODIMENT

The present invention will be further described in detail in combination with the accompanying drawings.

The same parts are indicated by the same reference number. It should be noted that the words “front”, “back”, “left”, “right”, “up” and “down” used in the following description refer to the directions in the drawings, and the words “bottom” and “top”, “inside” and “outside” refer to the directions towards or away from the geometric center of a specific part respectively.

### Example 1

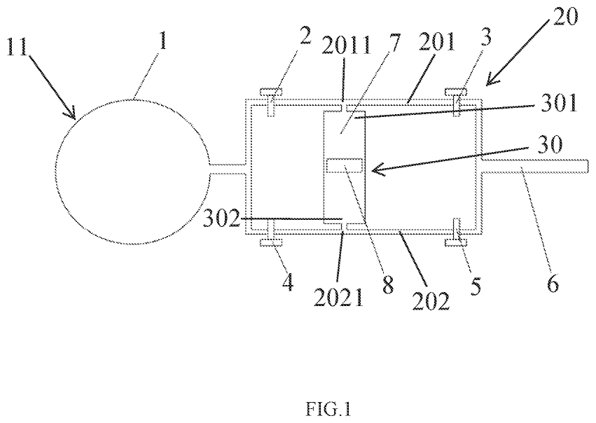

Referring to FIG. 1, a disposable wearable micro-flow continuous drug delivery system has an elastic drug storage cavity 1 for extruding and storing liquid drug, wherein one end of the elastic drug storage cavity 1 is communicated with a drug outlet pipe, wherein one end of the drug outlet pipe is communicated with a first micro-flow path and a second micro-flow path for shunting liquid drug, which is far away from the elastic drug storage cavity 1, wherein an adjusting mechanism is connected between the first micro-flow path and a second micro-flow path, wherein a first valve 2 and a second valve 3 are respectively provided at the surface of the first micro-flow path and adjacent to two different sides of the adjusting mechanism, a third valve 4 and a fourth valve 5 are respectively provided at the surface of the second micro-flow path and adjacent to the two different sides of the adjusting mechanism, wherein one end of the first micro-flow path is communicated with one end of the second micro-flow path, which are far away from the elastic drug storage cavity 1 and define an outlet 6, wherein the adjusting mechanism has a piston chamber 7 provided between the first micro-flow path and the second micro-flow path and a piston movably installed in the piston chamber 7.

Referring to FIG. 1, the first valve 2 is located on a side of the second valve 3, which is close to the elastic drug storage cavity 1, and the third valve 4 is located on a side of fourth valve 5, which is close to the elastic drug storage cavity 1.

Referring to FIG. 1, two ends of the adjusting mechanism are respectively communicated with the first micro-flow path and the second micro-flow path.

When in use, by controlling the first valve 2 and the fourth valve 5 to be opened, and closing the second valve 3 and the third valve 4 at the same time, when the liquid drug is pressurized in the elastic drug storage cavity 1 by an elastic force, the liquid drug flows out. Because the third valve 4 is closed and the first valve 2 is opened, the liquid drug from the elastic drug storage cavity 1 enters into the first micro-flow path where the first valve 2 located at, and then the liquid drug enters into the piston chamber 7 through the first valve 2 and the upper part thereof to push the piston 8 to move downward under the action of the liquid drug until the piston 8 arrives at the lower part of the piston chamber 7. During the piston 8 moves downward, the liquid drug under the piston 8 in the piston chamber 7 is extruded to flow from the fourth valve 5 to the outlet 6 to be injected into the human body through the outlet 6. The volume of the exported liquid drug is the volume of the piston chamber 7. At this time, the piston 8 is located at the bottom of the piston chamber 7.

When the control circuit changes the switch state to close the first valve 2 and the fourth valve 5 and open the second valve 3 and the third valve 4, the liquid drug would flow into the lower part of the piston chamber 7 through the second valve 3 and push the piston 8 to move upward the upper part of the piston chamber 7. At the same time, the liquid drug in the upper part of the piston chamber 7 is extruded to flow out of the outlet 6 through the second valve 3, and the volume of the exported liquid drug is the volume of the piston chamber 7, and the piston 8 is located at the top of the piston chamber 7. When the switch state of the four valves is alternately cycled, the liquid drug is provided continuously in a minimum volume of the volume of the piston chamber 7.

### Example 2

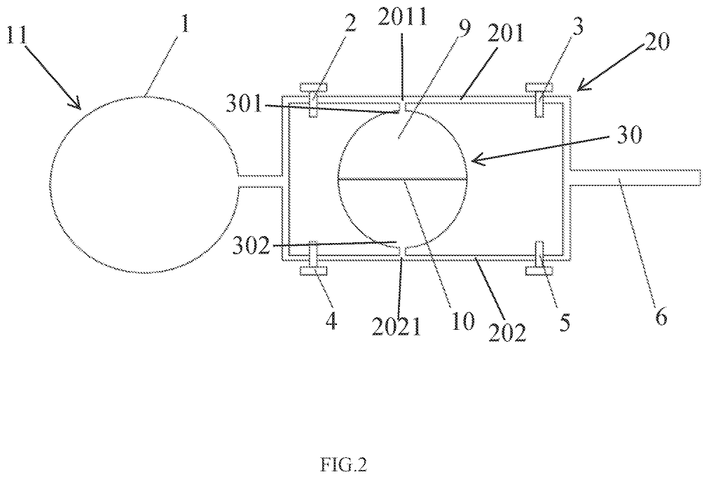

Referring to FIG. 2, a disposable wearable micro-flow continuous drug delivery system has an elastic drug storage cavity 1 for extruding and storing liquid drug, wherein one end of the elastic drug storage cavity 1 is communicated with a drug outlet pipe, wherein one end of the drug outlet pipe is communicated with a first micro-flow path and a second micro-flow path for shunting liquid drug, which is far away from the elastic drug storage cavity 1, wherein an adjusting mechanism is connected between the first micro-flow path and a second micro-flow path, wherein a first valve 2 and a second valve 3 are respectively provided at the surface of the first micro-flow path and adjacent to two different sides of the adjusting mechanism, a third valve 4 and a fourth valve 5 are respectively provided at the surface of the second micro-flow path and adjacent to the two different sides of the adjusting mechanism, wherein one end of the first micro-flow path is communicated with one end of the second micro-flow path, which are far away from the elastic drug storage cavity 1 and define an outlet 6, wherein the adjusting mechanism has a spherical cavity 9 provided between the first micro-flow path and the second micro-flow path and an elastic spacing layer 10 movably installed at the center of the spherical cavity 9.

Referring to FIG. 2, the first valve 2 is located on a side of the second valve 3, which is close to the elastic drug storage cavity 1, and the third valve 4 is located on a side of fourth valve 5, which is close to the elastic drug storage cavity 1.

Referring to FIG. 2, two ends of the adjusting mechanism are respectively communicated with the first micro-flow path and the second micro-flow path.

When in use, by controlling the first valve 2 and the fourth valve 5 to be opened, and closing the second valve 3 and the third valve 4 at the same time, when the liquid drug is pressurized in the elastic drug storage cavity 1 by an elastic force, the liquid drug flows out. Because the third valve 4 is closed and the first valve 2 is opened, the liquid drug from the elastic drug storage cavity 1 enters into the first micro-flow path where the first valve 2 located at, and then the liquid drug enters into the spherical cavity 9 through the first valve 2 and the upper part thereof to push the elastic spacing layer 10 to move downward under the action of the liquid drug until the elastic spacing layer 10 is attached on the bottom of the spherical cavity 9. During the elastic spacing layer 10 moves downwardly, the liquid drug under the elastic spacing layer 10 in the spherical cavity 9 is extruded to flow from the fourth valve 5 to the outlet 6 to be injected into the human body through the outlet 6. The volume of the exported liquid drug is the volume of the spherical cavity 9. At this time, the elastic spacing layer 10 is attached on the bottom of the spherical cavity 9.

When the control circuit changes the switch state to close the first valve 2 and the fourth valve 5 and open the second valve 3 and the third valve 4, the liquid drug would flow into the lower part of the spherical cavity 9 through the second valve 3 and push the elastic spacing layer 10 to move upward the upper part of the spherical cavity 9. At the same time, the liquid drug in the upper part of the spherical cavity 9 is extruded to flow out of the outlet 6 through the second valve 3, and the volume of the exported liquid drug is the volume of the spherical cavity 9, and the elastic spacing layer 10 is located at the top of the spherical cavity 9. When the switch state of the four valves is alternately cycled, the liquid drug is provided continuously in a minimum volume of the volume of the spherical cavity 9.

The basic principles, main features and advantages of the present invention are shown and described as above. Those skilled in the art should understand that the present invention is not limited by the above-mentioned embodiments and the above-mentioned embodiments and the description only describe the principle of the present invention. Without departing from the spirit and scope of the present invention, there would be various changes and improvements in the present invention, which should fall within the scope of the present invention. The scope of the present invention is defined by the attached claims and their equivalents.

1. A continuous drug delivery system, wherein the continuous drug delivery system comprises an elastic drug storage cavity for extruding and storing liquid drug, wherein one end of the elastic drug storage cavity is connected with a drug outlet pipe, wherein one end of the drug outlet pipe is connected with a first micro-flow path and a second micro-flow path for shunting liquid drug, which is far away from the elastic drug storage cavity, wherein an adjusting mechanism is connected between the first micro-flow path and the second micro-flow path, wherein a first valve and a second valve are respectively provided at the surface of the first micro-flow path and adjacent to two different sides of the adjusting mechanism, a third valve and a fourth valve are respectively provided at the surface of the second micro-flow path and adjacent to the two different sides of the adjusting mechanism, wherein one end of the first micro-flow path is communicated with one end of the second micro-flow path, which are far away from the elastic drug storage cavity and define an outlet, wherein the adjusting mechanism has a piston chamber provided between the first micro-flow path and the second micro-flow path and a piston movably installed in the piston chamber.

2. The continuous drug delivery system according to claim 1, wherein the first valve is located at one side of the second valve, which is close to the elastic drug storage chamber, and the third valve is located at one side of fourth valve, which is close to the elastic drug storage chamber.

3. (canceled)

4. The continuous drug delivery system according to claim 1, wherein two ends of the adjusting mechanism are respectively communicated with the first micro-flow path and the second micro-flow path.

5. A continuous drug delivery system, wherein the continuous drug delivery system comprises an elastic drug storage cavity for extruding and storing liquid drug, wherein one end of the elastic drug storage cavity is connected with a drug outlet pipe, wherein one end of the drug outlet pipe is connected with a first micro-flow path and a second micro-flow path for shunting liquid drug, which is far away from the elastic drug storage cavity, wherein an adjusting mechanism is connected between the first micro-flow path and the second micro-flow path, wherein a first valve and a second valve are respectively provided at the surface of the first micro-flow path and adjacent to two different sides of the adjusting mechanism, a third valve and a fourth valve are respectively provided at the surface of the second micro-flow path and adjacent to the two different sides of the adjusting mechanism, wherein one end of the first micro-flow path is communicated with one end of the second micro-flow path, which are far away from the elastic drug storage cavity and define an outlet, wherein the adjusting mechanism comprises a spherical cavity provided between the first micro-flow path and the second micro-flow path and an elastic spacing layer movably installed at the center of the spherical cavity.

6. The continuous drug delivery system according to claim 5, wherein the first valve is located at one side of the second valve, which is close to the elastic drug storage chamber, and the third valve is located at one side of fourth valve, which is close to the elastic drug storage chamber.

7. The continuous drug delivery system according to claim 5, wherein two ends of the adjusting mechanism are respectively communicated with the first micro-flow path and the second micro-flow path.

8. A continuous drug delivery system having an elastic bag, a drug outlet pipe, an adjusting mechanism, a first valve, a second valve, a third valve, a fourth valve and an outlet, wherein the elastic bag defines a drug storage cavity, the drug outlet pipe defines a first micro-flow path and a second micro-flow path, the adjusting mechanism defines a fluid chamber, wherein the fluid chamber has a first end and a second end, the first micro-flow path has a first communicating portion, and the second micro-flow path has a second communicating portion, wherein the first micro-flow path and the second micro-flow path are spaced from each other, the adjusting mechanism is arranged between the first micro-flow path and the second micro-flow path, the first end of the fluid chamber is communicated with the first communicating portion of the first micro-flow path, and the second end of the fluid chamber is communicated with the second communicating portion of the second micro-flow path, wherein the first micro-flow path is respectively communicated with the drug storage cavity and the outlet, the second micro-flow path is respectively communicated with the drug storage cavity and the outlet, wherein the first valve and the second valve are provided in the first micro-flow path, the third valve and the fourth valve are provided in the second micro-flow path, wherein the first communicating portion of the first micro-flow path is provided between the first valve and the second valve, the second communicating portion of the second micro-flow path is provided between the third valve and the fourth valve, and the adjusting mechanism further comprises a regulating element movably provided in the fluid chamber.

9. The continuous drug delivery system according to claim 8, wherein the fluid chamber is a spherical cavity provided between the first micro-flow path and the second micro-flow path and the regulating element is an elastic spacing layer movably installed at the center of the spherical cavity.

10. The continuous drug delivery system according to claim 8, wherein the fluid chamber is a piston chamber provided between the first micro-flow path and the second micro-flow path and the regulating element is a piston movably installed in the piston chamber.

11. The continuous drug delivery system according to claim 8, wherein the inner diameter of the first micro-flow path is different from that of the second micro-flow path.
